(19) United States
(12) Patent Application Publication
(10) Pub. No.: US 2020/0230378 A1
(43) Pub. Date: Jul. 23, 2020

(54) BALLOON CATHETER SYSTEM AND METHOD FOR OPTICAL TUMOR TREATMENT

(71) Applicant: OLYMPUS CORPORATION, Tokyo (JP)

(72) Inventor: Masahiro YOSHINO, Tokyo (JP)

(73) Assignee: OLYMPUS CORPORATION, Tokyo (JP)

(21) Appl. No.: 16/253,477

(22) Filed: Jan. 22, 2019

(51) Int. Cl. A61M 25/10; A61N 5/10

(52) U.S. Cl. CPC A61M 25/10181; A61N 5/1007; A61M 25/104; A61N 2005/1003; A61M 25/1002; A61M 2025/1088; A61N 5/1002

(57) ABSTRACT

A balloon catheter system includes: a light irradiation probe including, at a distal end portion of a fiber probe, a light irradiation portion for irradiating light; a balloon catheter including a shaft having a through hole through which the light irradiation probe is insertable, the through hole allowing liquid feeding, with the light irradiation probe being arranged, and a balloon provided to the shaft and configured to be expandable and contractable by a liquid injected into the balloon; and an injection apparatus including a measuring portion capable of measuring a quantity of the liquid, and an injection portion for injecting the liquid measured by the measuring portion into the balloon, and the balloon expands by the injected liquid, to thereby extend a wall of a region to be treated and retain the wall of the region to be treated in an extended state.

### BACKGROUND OF THE INVENTION

### 1. Field of the Invention

The present invention relates to a balloon catheter system and an optical tumor treatment method in which a light energy is applied through a balloon of a balloon catheter.

### 2. Description of Related Art

A lesion is resected in a surgical operation. In a transurethral resection of bladder tumor (hereinafter, referred to as TUR-Bt) for resecting a bladder tumor, for example, a perfusion liquid is flowed into the bladder, and resection of a lesion and collection of pieces of the resected lesion are performed, while ensuring a field of view of an endoscope. The bladder is expanded by the perfusion liquid, and the shape of the bladder is changed according to the content quantity of the perfusion liquid in the bladder.

The document “Urological Endoscopic Surgery Procedure Illustrated Reference Book—Support for independence in transurethral operation (Non-Patent Document 1) recites, on page 108, that when the TUR-Bt is performed, if the bladder is filled with 300 ml or more of the perfusion liquid, the wall of the bladder becomes thin, which causes a tensile force to be applied to the bladder, and if electricity is conducted for resection in such a state, the bladder is likely to be damaged.

Therefore, the flow rate of the perfusion liquid is required to be controlled for keeping the expanded state of the bladder constant in the TUR-Bt. On the other hand, Journal of Applied Clinical Medical Physics, Vol. 8, Nol, p55-68, Winter2007 (Non-Patent Document 2) recites the example of report that the average volume of the bladder immediately after urination is 118 milliliters.

In recent years, as an effective cancer treatment method, optical treatment methods such as PDT (photodynamic therapy), PIT (photoimmuno therapy), etc., have been studied.

U.S. Pat. No. 6,364,874 (referred to as Document 1), for example, discloses “Device for irradiating internal cavities of the body”. The apparatus in the Document 1 includes a flexible catheter made of a transparent material, an optical fiber designed to radially diffuse the light from a light source, and an expandable balloon fixed to one end of the catheter so as to surround a diffusion portion of the optical fiber.

The balloon is made of an elastomer material that diffuses light, and manufactured, in advance, by forming based on a shape of a cavity to be treated. FIG. 1 of Document 1 illustrates an instrument for irradiating the bronchus of a patient, and FIG. 2 of Document 1 illustrates an instrument for irradiating the uterus of a patient.

Each of the balloons is pressed against the wall of the cavity, and if the thickness of the surface of the balloon is constant, the pressure required for expansion is low. As a result, the balloon does not do damage on the tissues of the cavity.

### SUMMARY OF THE INVENTION

A balloon catheter system according to one aspect of the present invention includes: a light irradiation probe including, at a distal end portion of a fiber probe, a light irradiation portion configured to irradiate light; a balloon catheter including a shaft and a balloon, the shaft including a through hole through which the light irradiation probe is insertable, the through hole being configured to allow liquid feeding, with the light irradiation probe being arranged in the through hole, the balloon being fixed to one end portion of the shaft and configured to be expandable and contractable by a liquid injected through the through hole of the shaft; and an injection apparatus including a measuring portion configured to be capable of measuring a quantity of the liquid to be injected into the balloon, and an injection portion configured to inject the liquid measured by the measuring portion into the balloon through the through hole. The balloon expands by the liquid injected into the balloon, to thereby extend a wall of a region to be treated, and the expanded balloon retains the wall of the region to be treated in an extended state.

An optical tumor treatment method according to one aspect of the present invention includes: a balloon positioning step of positioning a balloon fixed to one end portion of a shaft of a balloon catheter in a bladder; a mucosa extending step of expanding the balloon by operating an injection portion of an injection apparatus to inject a liquid into the balloon through a through hole of the shaft and extending a wall of a region to be treated; a light irradiation portion positioning step of positioning a light irradiation portion of a light irradiation probe in the balloon extending the wall; and a light irradiation step of irradiating light from the light irradiation portion to a mucosa of the wall through the balloon.

### BRIEF DESCRIPTION OF THE DRAWINGS

FIG. 1 illustrates a balloon catheter system.

FIG. 2 illustrates steps in an optical treatment method.

FIG. 3 illustrates a balloon positioning step.

FIG. 4A relates to a mucosa extending step, and illustrates a state where normal saline is injected into a balloon.

FIG. 4B relates to the mucosa extending step, and illustrates a state where a part of an outer surface of the balloon comes into contact with a wall of a bladder.

FIG. 4C relates to the mucosa extending step, and illustrates a state where a tactile sensor provided on the outer surface of the balloon comes into contact with the mucosa of the wall.

FIG. 4D illustrates a balloon including, on the outer surface thereof, two tactile sensors.

FIG. 5 illustrates a light irradiation portion positioning step and a light irradiation step.

FIG. 6A illustrates a balloon catheter system including an image pickup probe in addition to an optical irradiation probe.

FIG. 6B illustrates a step of positioning a balloon, which is fixed to one end portion of a shaft including the image pickup probe, in the bladder.

FIG. 6C relates to the mucosa extending step, and illustrates a state where a part of the outer surface of the balloon comes into contact with the wall of the bladder.

FIG. 7A illustrates an endoscope system including the balloon catheter system and an endoscope into which the shaft of a balloon catheter is inserted.

FIG. 7B illustrates a step of positioning the balloon, which is fixed to the shaft inserted through a treatment instrument channel, in the bladder.

FIG. 7C relates to the mucosa extending step, and illustrates a state where the outer surface of the balloon comes into close contact with the wall of the bladder.

### DETAILED DESCRIPTION OF PREFERRED EMBODIMENTS

Hereinafter, embodiments of the present invention will be described with reference to drawings.

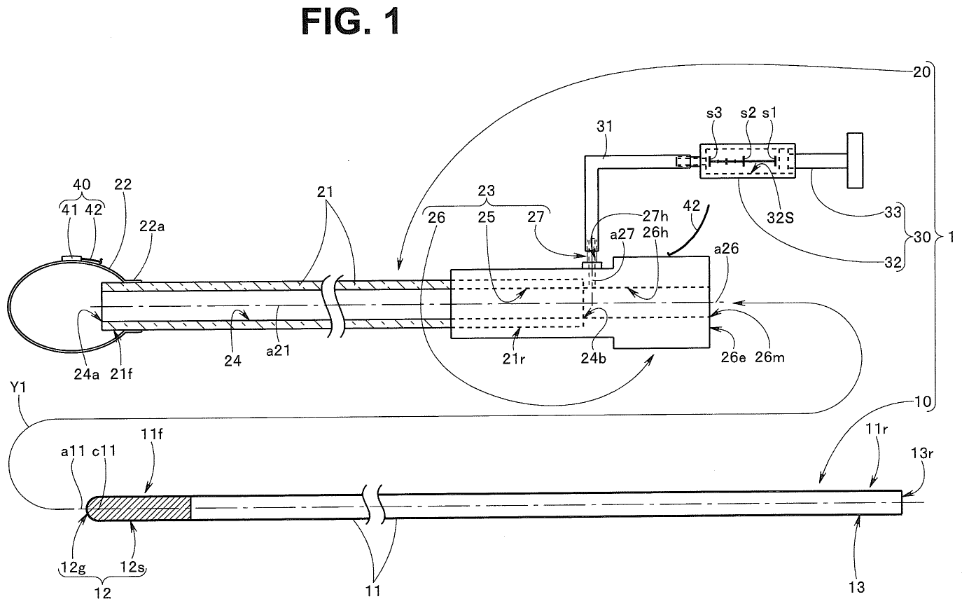

The reference sign 1 in FIG. 1 denotes a balloon catheter system. A balloon catheter system 1 is an optical treatment apparatus adapted to an optical tumor treatment such as PIT.

The balloon catheter system 1 includes a light irradiation probe 10, a balloon catheter 20, and an injection apparatus 30.

The light irradiation probe 10 includes a light irradiation portion 12 at a probe distal end portion 11f which is one end portion of an elongated fiber probe 11. The light irradiation portion 12 is a light irradiation body including a light diffusion function. The light irradiation portion 12 includes an irradiation portion peripheral surface 12s and an irradiation portion distal end surface 12g. The outer surface of the irradiation portion peripheral surface 12s has an annular shape. The outer surface of the irradiation portion distal end surface 12g has a hemispherical shape.

A probe proximal end portion 11r, which is located on an opposite side of the probe distal end portion 11f of the fiber probe 11, is a light source connection portion 13. The light source connection portion 13 is connected to a light source apparatus (not shown). The light source apparatus includes a light emitter (not shown). The light emitted from the light emitter is incident on a proximal end surface 13r, for example, which is a predetermined portion of the light source connection portion 13. The incident light is transmitted through the fiber probe 11, to be emitted radially (omnidirectionally) from the light irradiation portion 12.

Specifically, the light is emitted from the irradiation portion peripheral surface 12s radially relative to a fiber probe central axis all. On the other hand, the light is emitted from the irradiation portion distal end surface 12g radially relative to a center point c12.

The balloon catheter 20 includes a shaft 21 and a balloon 22. The reference sign 23 denotes a port, and the port is provided at the end portion of the shaft 21.

The shaft 21 is an elongated single-lumen tube having translucency. The shaft 21 includes one through hole 24 along a shaft axis a21. The shaft 21 is made of nylon, silicone, or Teflon (registered trademark).

The inner diameter of the through hole 24 provided in the shaft 21 is set to be larger than the outer diameter of the fiber probe 11 by a predetermined dimension. Therefore, the fiber probe 11 is insertable into the through hole 24.

The reference sign 24a denotes an opening for balloon which is a distal end side opening of the through hole 24. A proximal end side opening located on the opposite side of the opening for balloon 24a is an opening for port 24b.

The balloon 22 includes translucency and elasticity. The balloon 22 is a bag body having an opening on one side, and an opening side end portion 22a is a balloon fixing portion. The opening side end portion 22a of the balloon 22 is fixed, by welding or adhesion, to a predetermined position on the outer circumferential surface of a shaft distal end portion 21f of the shaft 21 so as to maintain liquid-tightness (water-tightness).

Therefore, the balloon 22 expands by a liquid being injected from the opening for balloon 24a into the balloon 22 through the through hole 24 of the shaft 21. In the present embodiment, the expanded shape of the balloon 22 is substantially spherical shape, and the balloon 22 is formed so as to expand from the opposite side of the opening side end portion 22a. In addition, the expanded balloon 22 contracts by the liquid inside the balloon 22 being discharged through the through hole 24.

A port 23 includes a shaft fixing hole 25, a probe port portion 26, and a liquid port portion 27. A shaft proximal end portion 21r, which is located on the opposite side of the shaft distal end portion 21f, is arranged in the shaft fixing hole 25. The shaft proximal end portion 21r is fixed to the shaft fixing hole 25 by welding or adhesion so as to hold watertightness.

The probe port portion 26 is provided on the proximal end portion side of the port 23. The liquid port portion 27 is provided so as to protrude from the side portion of the port 23.

The probe port portion 26 includes a probe insertion hole 26h. An insertion hole axis a26 of the probe insertion hole 26h coincides with the shaft axis a21 of the shaft 21. In addition, the inner diameter of the probe insertion hole 26h is approximately equal to the inner diameter of the through hole 24 so as to allow the fiber probe 11 to be inserted through the probe insertion hole.

The fiber probe 11 is inserted into the probe insertion hole 26h from an opening 26m on a proximal end surface 26e of the probe port portion 26, as shown by the arrow Y1. Then, the fiber probe 11 is passed through the probe insertion hole 26h, and thereafter introduced into the through hole 24 of the shaft 21.

Note that a retaining portion (not shown) is provided on the opening 26m side of the probe port portion 26. The retaining portion includes a function of a sealing cock configured to prevent the liquid to be supplied to the inside of the balloon 22 and to the inside of the through hole 24 from leaking outside and a function for retaining the arrangement position of the fiber probe 11.

The liquid port portion 27 includes a liquid hole 27h. A liquid feeding tube 31, which is extended from the injection apparatus 30, is detachably connected to the liquid port portion 27. In the present embodiment, the injection apparatus 30 is, what is called, an injector including a syringe 32 and a plunger 33.

A liquid hole axis a27 of the liquid hole 27h intersects the shaft axis a21 at a predetermined angle θ. The liquid hole axis a27 in the present embodiment is orthogonal to the shaft axis a21.

Note that the liquid port axis a27 may diagonally intersects the shaft axis a21 and may be extended from the side portion in the proximal end direction. In addition, the syringe 32 may be directly attached to the liquid port portion 27. In this case, the liquid port portion 27 serves as a syringe attaching portion to and from which the syringe 32 is attachable and detachable.

The syringe 32 is a cylindrical body having a liquid storage space 32S. The plunger 33 is arranged advanceably and retractably in the liquid storage space 32S so as to prevent the liquid from leaking outside from the liquid storage space 32S. The liquid stored in the liquid storage space 32S is passed through the inside of the liquid hole 27h and the inside of the through hole 24, and injected into the balloon 22 through the opening for balloon 24a, in accordance with the operation of the plunger 33 that functions as an injection portion.

The syringe 32 is provided with a plurality of scale marks and functions as a measuring portion. The syringe 32 is provided with scale marks s1, s2, and s3, for example. In the present embodiment, the first scale mark s1 notifies a user of a quantity of the liquid stored in advance in the syringe 32. The second scale mark s2 specifies the lower limit at the time when the liquid stored in the syringe 32 is injected into the balloon 22. The third scale mark s3 specifies the upper limit at the time when the liquid stored in the syringe 32 is injected into the balloon 22.

The reference sign 40 denotes a contact detection portion, and the contact detection portion 40 detects that the outer surface of the balloon 22 comes into contact with the wall of a region to be treated. The contact detection portion 40 includes a tactile sensor 41 having a function for detecting the pressure, the reflection of light, the capacity change between the electrodes, and the like, and a signal line 42 extended from the tactile sensor 41.

In the balloon catheter system 1 according to the present embodiment, when the tactile sensor 41 comes into contact with the wall of the region to be treated, a detection signal is outputted through the signal line 42, to cause a buzzer (not shown) to sound, for example. As a result, the operator is capable of judging that the wall 2w is brought into a state extended by the balloon 22.

Note that the detection signal may be outputted wirelessly from the tactile sensor 41.

The tactile sensor 41 is fixed to one portion which is a predetermined position on the outer surface of the balloon 22. The signal line 42 is extended from the tactile sensor 41 and arranged on the outer surfaces of the shaft 21 and the port 23, along the shaft axis a21 and the insertion hole axis a26. The signal line 42 is extended from the side portion on the proximal end side of the port 23, for example.

In the present embodiment, the injection apparatus 30 is an injector including the syringe 32 and the plunger 33. However, the injection apparatus 30 may be a pump-type fluid supply apparatus (not shown), for example. In such a fluid supply apparatus, a pump (not shown) is driven by the operation of a pump driving switch (not shown) and the liquid is injected into the balloon 22 while the quantity of the liquid is being measured, to cause the balloon 22 to expand. Therefore, the pump is the injection portion. The measurement of the injection quantity of the liquid into the balloon 22 is performed by a flow rate sensor, which functions as a measuring portion, provided to the liquid feeding tube 31.

Hereinafter, description will be made on the optical tumor treatment method using the balloon catheter system 1.

The balloon catheter system 1 according to the present embodiment is used for treating the tumor of the bladder. Therefore, the balloon catheter 20 is disposable catheter for urinary organs, and the balloon 22 is a balloon for the bladder which is arranged in the bladder as a region to be treated.

The volume of the fluid storage space 32S of the syringe 32 in the injection apparatus 30 corresponds to the volume of the bladder. That is, the second scale mark s2 of the syringe 32 is provided for notifying that the liquid stored in the syringe 32 up to the scale mark s1 is injected into the balloon 22 by 100 milliliters. The third scale mark s3 of the syringe 32 is provided for notifying that the liquid stored in the syringe 32 up to the scale mark s1 is injected into the balloon 22 by 300 milliliters.

The quantity of 100 milliliters is a value supposed based on the average volume of the bladder recited in the above-described non-patent document 2. On the other hand, the quantity of 300 milliliters is a value determined under consideration of safety based on the recitation of the above-described non-patent document 1. That is, in the treatment of the tumor of the bladder, the injection quantity of the liquid into the balloon 22 is set to be 100 milliliters or more but 300 milliliters or less.

Note that a medical staff member arranges the fiber probe 11 in advance in the through hole 24 of the shaft 21. In addition, the medical staff member connects, in advance, the syringe 32 in which normal saline is stored up to the first scale mark s1 and the liquid port portion 27 by the liquid feeding tube 31.

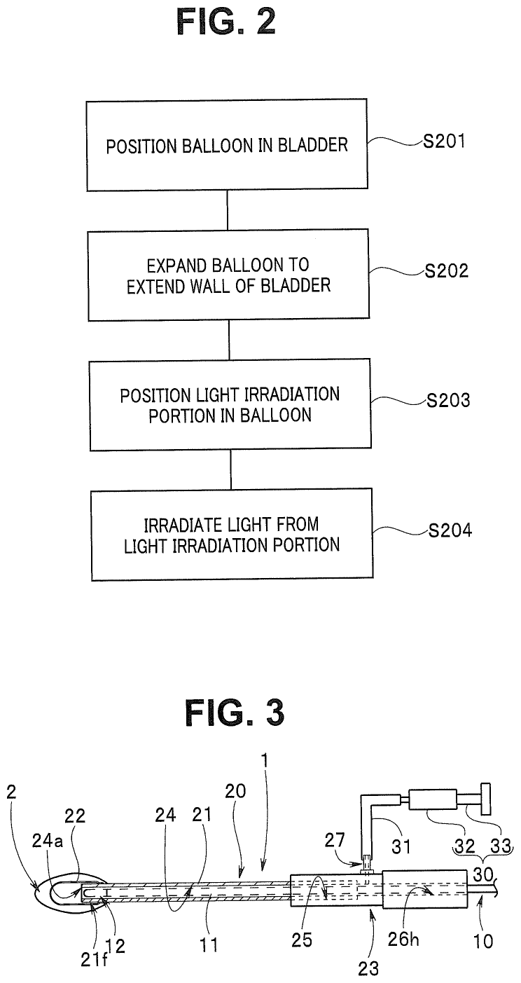

The medical staff member administers, in advance, a medicinal agent that reacts to the light emitted from the light irradiation portion 12 to the patient via intravenous injection, before starting the optical treatment of the bladder by using the balloon catheter system 1. The treatment is started after a predetermined time period has elapsed. The treatment includes a balloon positioning step S201, a mucosa extending step S202, a light irradiation portion positioning step S203, and a light irradiation step S204, as shown in FIG. 2.

The mucosa extending step S202 includes a first expansion retaining step. In the first expansion retaining step, when the tactile sensor 41 provided on the outer surface of the balloon 22 detects that the outer surface of the balloon comes into contact with the wall 2w, the operator stops the injection of the normal saline into the balloon 22 by the operation of the plunger (hereinafter, recited as an injection portion) 33, to retain the balloon in the expanded state.

In the balloon positioning step S201, the operator positions the balloon 22, which is fixed to the one end portion of the shaft 21 of the balloon catheter 20, in the bladder 2, as shown in FIG. 3. The reference sign 14 in FIG. 3 denotes a positioning colored portion. The colored portion 14 is a circumferential portion provided, in advance, at a predetermined position of the shaft 21. The position of the distal end side of the colored portion 14 is aligned with the position of the proximal end surface 26e of the probe port portion 26, to thereby allow the light irradiation portion 12 to be arranged at the predetermined position in the through hole 24.

Note that the position of the proximal end side of the colored portion 14 is aligned with the position of the proximal end surface 26e of the probe port portion 26, to thereby allow the light irradiation portion 12 to be arranged at the predetermined position in the expanded balloon 22 (see FIG. 5).

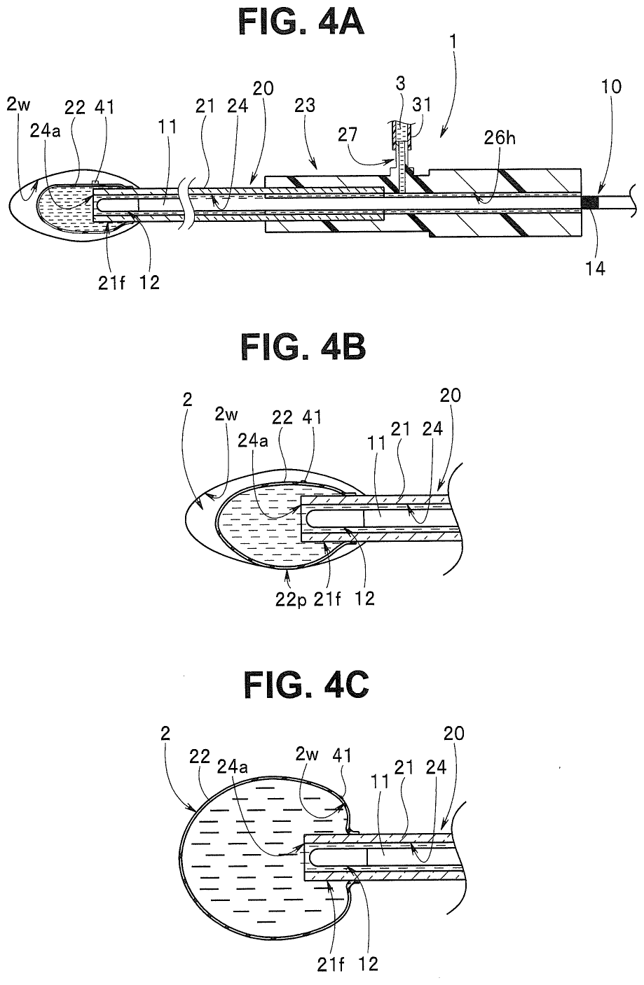

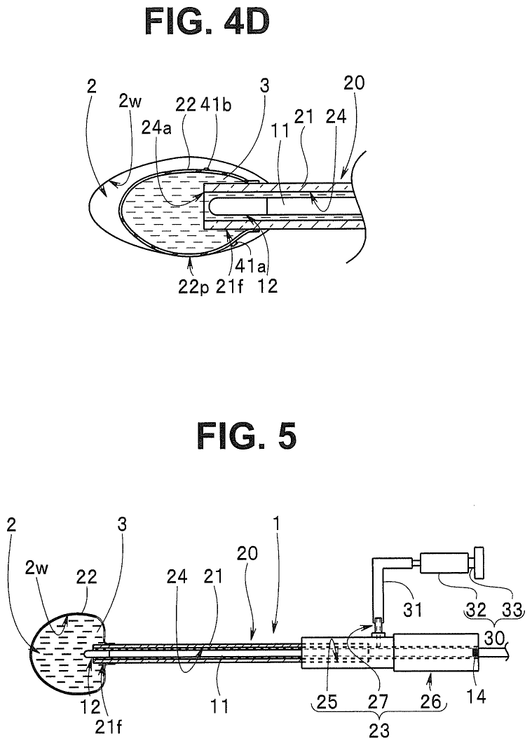

In the mucosa extending step S202, the operator operates the injection portion 33 of the injection apparatus 30. In accordance with the movement of the injection portion 33, the normal saline in the liquid storage space 32S is injected into the balloon 22 through the through hole 24 of the shaft 21. Then, the inside of the balloon 22, the inside of the through hole 24, and the inside of the probe insertion hole 26h are filled with the normal saline 3, as shown in FIG. 4A.

After that, the normal saline 3 is further injected into the balloon 22, to thereby cause the balloon 22 to start to expand. The operator confirms whether the residual quantity of the normal saline 3 in the liquid storage space 32S reaches the vicinity of the second scale mark s2 or reaches between the second scale mark s2 and the third scale mark s3, while performing the injection operation with the injection portion 33.

The operator continues the injection of the normal saline 3, which causes a part of the outer surface of the balloon 22 (see the reference sign 22p in FIG. 4B) to come into contact with the wall 2w of the bladder 2, as shown in FIG. 4B. Then, the operator further injects the normal saline 3 into the balloon 22, to thereby cause the balloon 22 to expand and cause the contacting surface between the outer surface and the wall 2w to increase.

As shown in FIG. 4C, the tactile sensor 41 provided on the outer surface of the balloon 22 comes into contact with the mucosa of the wall 2w, as shown in FIG. 4C. Then, a detection signal is outputted from the tactile sensor 41, and the buzzer rings. Upon recognizing the buzzer sound, the operator stops the operation of the injection portion 33, and confirms whether or not the buzzer sound continues to ring. While the buzzer sound can be heard, the outer surface of the balloon 22 contacts substantially the entire surface of the wall 2w, to thereby retain the wall 2w in the extended state.

In the light irradiation portion positioning step S203, the operator advances the light irradiation portion 12 of the fiber probe 11 in the through hole 24, while confirming the buzzer sound. This allows a part of 12g and a part of 12s of the light irradiation portion 12 to be protruded from the opening for balloon 24a of the through hole 24 and arranged in the balloon 22 which causes the bladder 2 to expand, as shown in FIG. 5. As a result, the fiber probe 11 is positioned and retained by the retaining portion, not shown.

In the light irradiation step S204, the operator causes the light to irradiate from the light irradiation portion 12, while confirming the buzzer sound. That is, the operator operates a foot switch (not shown), for example, for a predetermined time period. Then, the light is emitted from the light emitter of the light source apparatus, and the light is passed through the fiber probe 11, emitted from the light irradiation portion 12, passed through the balloon 22, and irradiated to the mucosa of the wall 2w.

After the end of the light irradiation step, the operator causes the normal saline 3 to discharge from the inside of the balloon 22, to contract the balloon 22, and extracts the balloon catheter 20 to the outside of the body.

Thus, according to the present embodiment, the bladder 2 can be expanded while gradually expanding the balloon 22 by injecting the liquid such as the normal saline 3 into the balloon 22. When the tactile sensor 41 provided on the outer surface of the balloon 22 comes into contact with the mucosa of the wall 2w of the bladder 2, the injection of the normal saline 3 into the balloon 22 is stopped, to enable both the bladder 2 and the balloon 22 to be retained in the expanded state.

In this expansion-retained state, the wall 2w of the bladder 2 is extended by the outer surface of the balloon 22 that expands and comes into contact with the wall 2w. Judgment of the extended state is enabled by confirming the buzzer sound which is sounded by the tactile sensor 41 coming into contact with the wall 2w of the bladder 2. Therefore, the operator causes the light irradiation portion 12 of the fiber probe 11 to emit light omnidirectionally, while confirming the buzzer sound, to thereby enable the mucosa of the wall 2w extended through the balloon 22 to be irradiated with the light.

In addition, the syringe 32 is provided with the scale marks s2 and s3 that respectively notify the lower limit and the upper limit of the normal saline 3 to be injected into the balloon 22. As a result, insufficient supply and excessive supply of the normal saline 3 into the balloon 22 can be prevented. Specifically, it is possible to surely prevent the state where the wall of the bladder becomes thin by the excessive supply of the normal saline and a tensile force is applied to the bladder.

In addition, when the buzzer sounds before the residual quantity of the normal saline reaches the scale mark s2, it is possible to easily judge that a failure occurs in the balloon positioning step S201. In this case, the balloon positioning step is performed again to eliminate the failure.

In the above-described embodiment, the tactile sensor 41 is fixed to one portion on the outer surface of the balloon 22. However, the number of the tactile sensor 41 is not limited to one, but tactile sensors may be provided at a plurality of portions. Specifically, as shown in FIG. 4D, tactile sensors 41a, 41b are provided on the outer surface of the balloon 22. In such a configuration, the buzzer sounds when a detection signal is outputted from each of the tactile sensors 41. As a result, the contact between the outer surface of the balloon 22 and the wall 2w can be determined with high precision.

Note that the positions where the tactile sensors 41 are provided are different depending on the expanded shape, the expanding start position and the like of the balloon 22, and three or more tactile sensors 41 may be provided. In addition, the operator may be notified that the wall 2w is extended by the balloon 22 by lighting or blinking of a lamp, vibration of a vibrator, and the like, instead of sounding of the buzzer.

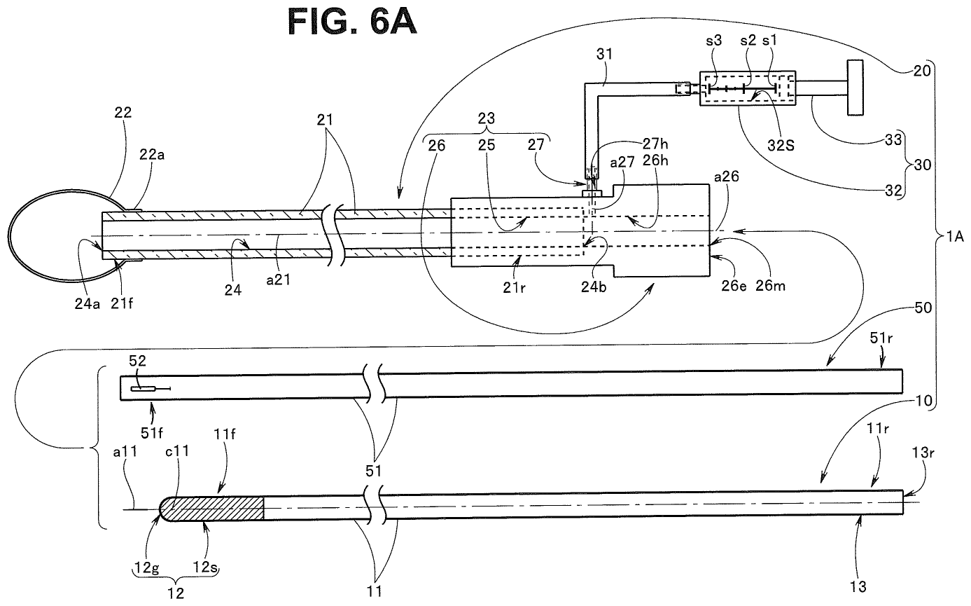

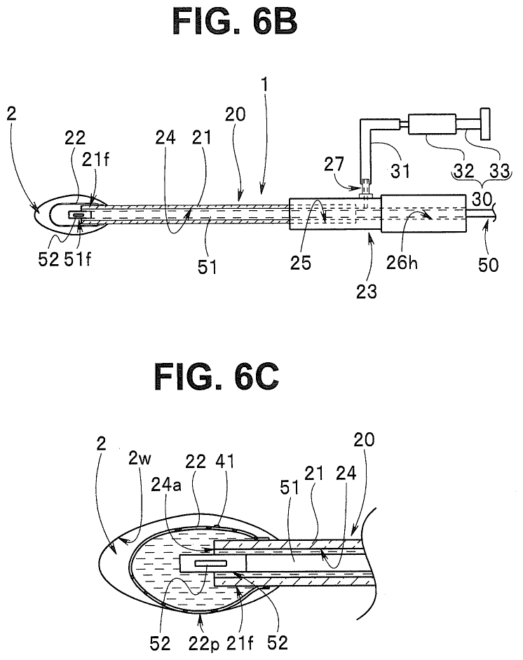

In the above-described embodiment, the contact detection portion 40 is the tactile sensor 41 provided on the outer surface of the balloon 22. However, the contact detection portion 40 is not limited to the tactile sensor 41, and may be an image pickup probe including an image sensor. In the present embodiment, a balloon catheter system 1A includes an image pickup probe 50, in addition to the light irradiation probe 10, as shown in FIG. 6A.

The image pickup probe 50 includes an image pickup device 52 and a light-emitting element (not shown) at an insertion portion distal end portion 51f which is one end portion of an elongated insertion portion 51. An insertion portion proximal end portion 51r, which is located on the opposite side of the insertion portion distal end portion 51f of the insertion portion 51, is connected to the camera control unit (not shown). The camera control unit is configured to convert an image pickup signal obtained by the image pickup device 52 to generate a video signal and output the generated video signal to a display apparatus (not shown). As a result, an observation image captured by the image pickup device 52 is displayed on the screen of the display apparatus. Note that an observation window and an illumination window, which are not shown, are provided on the distal end surface or a side surface of the insertion portion distal end portion 51f.

When performing an optical treatment of the bladder by using the balloon catheter system 1A, the medical staff member arranges the insertion portion 51, instead of the fiber probe 11, in the through hole 24 of the shaft 21. The outer diameter of the insertion portion 51 is set to be equal to or slightly smaller than the outer diameter of the fiber probe 11.

In addition, the mucosa extending step S202 includes a second expansion retaining step, instead of the first expansion retaining step. In the second expansion retaining step, the operator observes the image through the balloon 22, which is displayed on the screen of the display apparatus, to confirm that the balloon 22 is in contact with the wall 2w, and stops the injection of the normal saline into the balloon 22. As a result, the balloon 22 is retained in the expanded state.

Note that the tumor region to be treated is the bladder. The medical staff member connects the syringe 32 and the liquid port portion 27 with the liquid feeding tube 31. In addition, the medical staff member administers a medicinal agent that reacts to the light emitted from the light irradiation portion 12 to the patient via intravenous injection. Furthermore, the retaining portion on the opening 26m side of the probe port portion 26 includes a function for retaining the arranging positions of the fiber probe 11 and the insertion portion 51 and a function of a sealing cock configured to prevent the liquid to be supplied to the balloon 22 and to the inside of the through hole 24 from leaking outside.

In the balloon positioning step S201, the operator positions the balloon 22 fixed to the one end portion of the shaft 21 of the balloon catheter 20 in the bladder 2, as shown in FIG. 6B.

After that, in the mucosa extending step S202, the operator injects the normal saline 3 stored in the liquid storage space 32S into the balloon 22 in the similar manner as described above. As described above, the balloon 22, the through hole 24, and the probe insertion hole 26h are filled with the normal saline 3, and thereafter the balloon 22 starts to expand.

The operator continues the injection operation while performing injection operation by the injection portion 33, observing the image displayed on the screen of the display apparatus, and confirming the residual quantity in the liquid storage space 32S. Then, as shown in FIG. 6C, a part of the outer surface of the balloon 22 (see the reference sign 22p in FIG. 6C) comes into contact with the wall 2w of the bladder 2, and the image of the mucosa of the wall to which the outer surface of the balloon contacts through the balloon 22 is displayed on the screen of the display apparatus.

The operator observes a change in the contact area between the balloon 22 and the wall 2w while repeatedly performing the injection operation of the normal saline 3 and the rotation operation of the insertion portion 51 around the axis. Then, the operator confirms the image of the mucosa of the wall 2w extended through the balloon 22 by the outer surface of the balloon 22 coming into close contact with substantially the entire surface of the wall 2w, and at the same time, stops the operation of the injection portion 33. As a result, the outer surface of the balloon 22 retains the wall 2w in the extended state while in contact with the wall 2w.

After that, the procedure shifts to the light irradiation portion positioning step S203. After extracting the insertion portion 51 from the through hole 24 of the shaft 21, the operator inserts the fiber probe 11 of the light irradiation probe 10 into the through hole 24, to arrange the light irradiation portion 12 in the balloon 22 which is expanded while in contact with the wall 2w of the bladder 2, as shown in FIG. 5. Then, the procedure shifts to the above-described light irradiation step S204.

After the end of the light irradiation step, the operator causes the normal saline 3 to discharge from the inside of the balloon 22 to contract the balloon 22, and extracts the balloon catheter 20 to the outside of the body.

Thus, the balloon catheter system 1A according to the present embodiment includes the image pickup probe 50, in addition to the light irradiation probe 10. When confirming the image of the mucosa of the wall 2w extended through the balloon 22, which is captured by the image pickup device 52 of the image pickup probe 50 and displayed on the screen, the operator stops the injection of the normal saline 3 into the balloon 22. This enables the bladder 2, together with the balloon 22, to be retained in the expanded state where light irradiation is possible.

Note that, according to the above-described balloon catheter system 1A, when the procedure shifts to the light irradiation portion positioning step S203, the operation of extracting the insertion portion 51 from the through hole 24 of the shaft 21 and the operation of inserting the fiber probe 11 into the through hole 24 are performed. Such operations are required, since the shaft 21 is a single-lumen tube.

In view of the above, the shaft 21 is configured as a double-lumen tube including an insertion portion hole (not shown) as a first tube through hole through which the insertion portion 51 is insertable, and a fiber probe hole (not shown) as a second tube through hole through which the fiber probe 11 is insertable. Such a configuration enables the procedure to smoothly shift to the light irradiation portion positioning step S203 and then shift to the light irradiation step S204, with the insertion portion 51 arranged in the insertion portion hole, without the need for extracting the insertion portion 51 from the insertion portion hole.

In addition, according to the configuration, the operator is capable of reconfirming the image captured by the image pickup device 52 of the image pickup probe 50 in the light irradiation portion positioning step S203, and thereafter shifting the procedure to the light irradiation step S204.

In the above-described embodiment, the contact detection portion 40 is the image pickup probe 50 including the image pickup device 52. However, the contact detection portion may be an image pickup device 52A of an endoscope 60 including a treatment instrument channel through which the shaft 21 of the balloon catheter 20 is inserted.

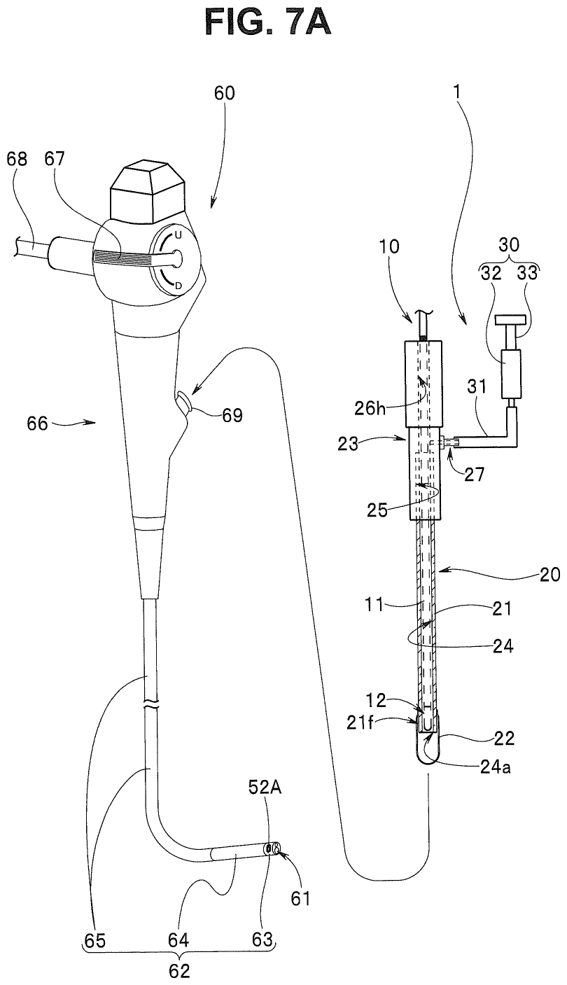

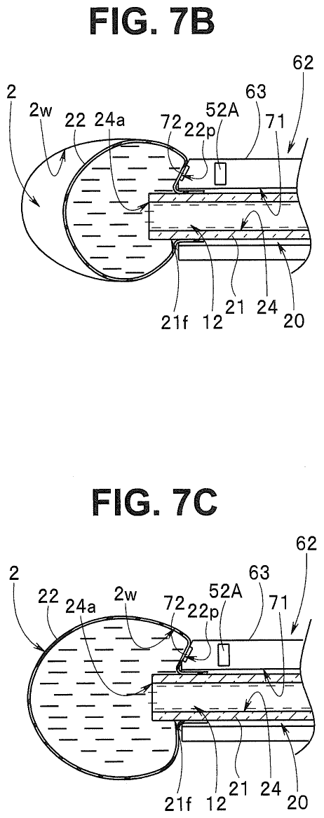

The endoscope system shown in FIG. 7A includes the endoscope 60, and configured such that the shaft 21 of the balloon catheter 20 is introduced from a treatment instrument opening 61 of the endoscope 60 into the treatment instrument channel to be led out into the bladder, for example. The reference signs 62, 63, 64, and 65 denote an endoscope insertion portion, an endoscope distal end portion, an endoscope bending portion, and an endoscope flexible tube portion, respectively. The endoscope bending portion 64 bends in accordance with the operation of a bending lever 67 provided at an endoscope operation portion 66. The reference signs 68 and 69 denote a universal cord and a treatment instrument insertion port, respectively. A treatment instrument opening 61 and a treatment instrument insertion port 69 communicate with the treatment instrument channel. The universal cord 68 incorporates a signal line extended from the image pickup device 52A.

In the present embodiment, the distal end surface of the endoscope distal end portion 63 of the endoscope insertion portion 62 is arranged in the vicinity of the bladder. Then, in the balloon positioning step S201, the operator positions the balloon 22, which is fixed to the shaft 21 of the balloon catheter 20 inserted through the treatment instrument channel 71, in the bladder 2, while observing the image of the balloon 22, as shown in FIG. 7B.

After that, the operator injects the normal saline 3 into the liquid storage space 32S in the mucosa extending step S202, similarly as described above. After the inside of the balloon 22, the inside of the through hole 24, and the inside of the probe insertion hole 26h are filled with the normal saline 3, the balloon 22 starts to expand.

The operator continues the injection operation while performing injection operation by the injection portion 33, observing the image displayed on the screen of the display apparatus, and confirming the residual quantity in the liquid storage space 32S. Then, a part (see the reference sign 22p in FIGS. 7B, 7C) of the outer surface of the balloon 22 comes into close contact with an observation window 72 of the endoscope 60, and the image of the balloon 22 or the image of the mucosa of the wall 2w to which the outer surface of the balloon contacts through the balloon 22 is displayed on the screen of the display apparatus.

The operator observes the change in the contact area between the balloon 22 and the wall 2w while repeatedly performing the injection operation of the normal saline 3 and the operation of the endoscope bending portion 64. Then, upon confirming the image of the mucosa of the wall 2w extended through the balloon 22 by the outer surface of the balloon 22 coming into close contact with substantially entire surface of the wall 2w as shown in FIG. 7C, the operator stops the operation of the injection portion 33. As a result, the outer surface of the balloon 22 retains the wall 2w in the extended state while in contact with the wall 2w.

After that, the procedure shifts to the light irradiation portion positioning step S203. At this time, the operator arranges the light irradiation portion 12 in the balloon 22, which is expanded while in close contact with the wall 2w of the bladder 2 as shown in FIG. 5, while confirming the image, to shift the procedure to the above-described light irradiation step S204.

After the end of the light irradiation step, the operator extracts the endoscope insertion portion 62 to the outside of the body.

Thus, in the present embodiment, the operator inserts the shaft 21 of the balloon catheter 20 of the balloon catheter system 1 into the treatment instrument channel 71 of the endoscope 60, to position the shaft 21 in the bladder. After that, the operator confirms, through the balloon 22 or directly, the contacting state between the outer surface of the balloon 22 and the wall 2w with the image pickup device 52A of the endoscope 60, while causing the balloon 22 to expand by injecting the normal saline 3 into the balloon 22.

Upon confirming the closely contacting state between the balloon 22 and the wall 2w based on the displayed image, the operator stops the injection of the normal saline 3 into the balloon 22. As a result, the bladder 2, together with the balloon 22, can be retained in the expanded state where light irradiation is possible, as described above.

The present invention is not limited to the above-described embodiments, and various changes, modifications, and the like are possible in a range without changing the gist of the present invention.

What is claimed is:

1. A balloon catheter system comprising:
a light irradiation probe including, at a distal end portion of a fiber probe, a light irradiation portion configured to irradiate light;
a balloon catheter including a shaft and a balloon, the shaft including a through hole in which the light irradiation probe is arrangeable, the through hole being configured to allow liquid feeding, with the light irradiation probe being arranged in the through hole, the balloon being fixed to one end portion of the shaft and configured to be expandable and contractable by a liquid injected through the through hole of the shaft; and
an injection apparatus including a measuring portion configured to be capable of measuring a quantity of the liquid to be injected into the balloon, and an injection portion configured to inject the liquid measured by the measuring portion into the balloon through the through hole,
wherein the balloon expands by the liquid injected into the balloon, to thereby extend a wall of a region to be treated, and the expanded balloon retains the wall of the region to be treated in an extended state.

2. The balloon catheter system according to claim 1, further comprising a contact detection portion configured to detect that the wall is extended by an outer surface of the balloon.

3. The balloon catheter system according to claim 2, wherein the contact detection portion is a tactile sensor arranged on the outer surface of the balloon and configured to detect that the wall is extended by contacting a mucosa of the wall.

4. The balloon catheter system according to claim 3, wherein the tactile sensor is provided on at least one portion of the outer surface of the balloon.

5. The balloon catheter system according to claim 2, wherein the contact detection portion is an image pickup device configured to pick up an image of the balloon and an image of the wall.

6. The balloon catheter system according to claim 5, wherein the image pickup device is arranged in the balloon and configured to pick up the image of the wall through the balloon.

7. The balloon catheter system according to claim 5, wherein the image pickup device is included in an endoscope comprising a treatment instrument channel through which the balloon catheter is insertable.

8. The balloon catheter system according to claim 1, wherein the measuring portion defines a lower limit and an upper limit of the liquid to be injected into the balloon.

9. The balloon catheter system according to claim 8, wherein when the balloon is a balloon for bladder configured to be arranged in the bladder, a quantity of the liquid to be injected into the balloon is not less than 100 milliliters but not more than 300 milliliters.

10. An optical tumor treatment method comprising:
a balloon positioning step of arranging a balloon fixed to one end portion of a shaft of a balloon catheter in a region to be treated;
a mucosa extending step of expanding the balloon by operating an injection portion of an injection apparatus to inject a liquid into the balloon through a through hole of the shaft and extending a wall of the region to be treated;
a light irradiation portion positioning step of arranging a light irradiation portion of a light irradiation probe in the balloon extending the wall, and
a light irradiation step of irradiating light from the light irradiation portion to a mucosa of the wall through the balloon.

11. The optical tumor treatment method according to claim 10, wherein the mucosa extending step further comprises a first expansion retaining step of retaining the balloon in an expanded state by stopping injection of the liquid into the balloon by the injection portion when a tactile sensor provided on an outer surface of the balloon detects that the outer surface of the balloon comes into contact with the wall.

12. The optical tumor treatment method according to claim 10, wherein the mucosa extending step further comprises a second expansion retaining step of retaining the balloon in an expanded state by stopping injection of the liquid into the balloon by the injection portion when a contact between the outer surface of the balloon and the wall is visually recognized based on an image picked up by an image pickup device.

13. The optical tumor treatment method according to claim 12, wherein the image pickup device is provided in an image pickup probe configured to be arranged in the balloon.

14. The optical tumor treatment method according to claim 12, wherein the image pickup device is provided in an endoscope including a treatment instrument channel through which the balloon catheter is insertable.

15. The optical tumor treatment method according to claim 10, wherein when the balloon is a balloon for bladder configured to be arranged in the bladder, not less than 100 milliliters but not more than 300 milliliters of liquid is injected into the balloon.
